(19) United States
(12) Patent Application Publication
(10) Pub. No.: US 2022/0257406 A1
(43) Pub. Date: Aug. 18, 2022

(54) BODILY FLUID COLLECTION DEVICE AND METHODS

(71) Applicant: Shari Goldman Snow, Chicago, IL (US)

(72) Inventor: Shari Goldman Snow, Chicago, IL (US)

(21) Appl. No.: 17/670,152

(22) Filed: Feb. 11, 2022

Related U.S. Application Data
(60) Provisional application No. 63/149,211, filed on Feb. 12, 2021.

(51) Int. Cl. A61F 5/44; A61F 5/455

(52) U.S. Cl. CPC A61F 5/4407; A61F 5/4553

(57) ABSTRACT

A bodily fluid collection device is described and comprises an annular rim, a film reservoir, and a drawstring. Methods of collecting bodily fluids are also described.

### PRIORITY

This application claims the benefit of U.S. Ser. No. 63/149,211, filed Feb. 12, 2021, which is incorporated by reference herein in its entirety.

### FIELD OF THE DISCLOSURE

The disclosure generally relates to feminine hygiene products and bodily fluid collection.

### BACKGROUND OF THE DISCLOSURE

Menstrual flow and vaginal secretions from the uterus of female humans have traditionally been collected using an absorbent material, such as tampons or pads. However, tampons have limited absorptive capacity, can leak, and must be changed frequently. Failing to remove tampons within eight hours can cause Toxic Shock Syndrome, a potentially fatal medical condition. Externally applied pads require frequent changing, are bulky and uncomfortable, and can leak. Other devices such as menstrual cups and discs have also been developed over the years. However, cups and discs can be uncomfortable and difficult to remove resulting in heavy leakage of bodily fluids and staining of clothes. Menstrual cups and discs have limited filling capacity, and need to be cleansed upon removal, which is messy, and may cause embarrassment if in a public setting. As a result, improvements in the field of bodily fluid collection are needed.

### SUMMARY OF THE DISCLOSURE

Systems and methods directed to a bodily fluid collection device are disclosed.

A bodily fluid collection device is disclosed. The bodily fluid collection device can comprise a film reservoir for receiving a volume of bodily fluids, an annular rim attached above the film reservoir, and a drawstring configured to seal the film reservoir. The bodily fluid collection device can be inserted into a body part, such as a vagina or vaginal canal, and removed from an orifice, such as a vaginal opening, without needing to use fingers internally to remove the device from the body part. The bodily fluid collection device can be sealable or spill proof such that when it is removed from the body part through the orifice, it can be in a closed state.

In an embodiment, a bodily fluid collection device, can include: a film reservoir for receiving a volume of bodily fluids; an annular rim attached above the film reservoir: and a drawstring configured to seal the film reservoir.

In some embodiments, the film reservoir can be partially removably attached to the annular rim. In some embodiments, the bodily fluid collection device can further include a sleeve affixed to an upper portion of the film reservoir, wherein the sleeve houses the drawstring. In some embodiments, a space between the sleeve and the annular rim comprises perforations circling a diameter of the film reservoir. In some embodiments, the annular rim can have an opening that receives a portion of the drawstring. In some embodiments, the drawstring can include a knot. In some embodiments, the film reservoir can expand and stretches while collecting bodily fluids. In some embodiments, the bodily fluid collection device can collect vaginal discharge.

In another embodiment, a method of collecting bodily fluids can include: inserting a bodily fluid collection device into a vagina, wherein the bodily fluid collection device comprises a film reservoir for receiving a bodily fluid, an annular rim attached above the film reservoir, and a drawstring configured to seal the film reservoir; collecting a volume of bodily fluids within the film reservoir; pulling a distal end of the drawstring to seal the film reservoir comprising a volume of bodily fluids within the film reservoir; and removing the bodily fluid collection device from an orifice with the film reservoir in a closed state.

In some embodiments, pulling the distal end of the drawstring to seal the film reservoir including a volume of bodily fluids within the film reservoir can include cinching the film reservoir closed with the drawstring. In some embodiments, pulling the distal end of the drawstring to seal the film reservoir including a volume of bodily fluids within the film reservoir can include severing a portion of the film reservoir with the drawstring, wherein pulling the drawstring constricts the film reservoir. In some embodiments, the method of collecting bodily fluid can further include housing the drawstring in a sleeve affixed to an upper portion of the film reservoir.

These and other objects, features, and advantages of the present disclosure will become more readily apparent from the attached drawings and the detailed description of the preferred embodiments, which follow.

### BRIEF DESCRIPTION OF THE DRAWINGS

FIG. 1 is a perspective view of an exemplary embodiment the bodily fluid collection device as described in the present disclosure;

FIG. 2 is an alternate view of an exemplary embodiment the bodily fluid collection device as described in the present disclosure;

FIG. 3A is an isometric view exemplary embodiment of the bodily fluid collection device as described in the present disclosure;

FIG. 3B is a cross-sectional isometric view of exemplary embodiment the bodily fluid collection device as described in the present disclosure; and

FIG. 4 is an isometric view of an exemplary embodiment of the bodily fluid collection device not comprising perforations as described in the present disclosure.

### DETAILED DESCRIPTION

The invention is defined by the claims. The description is exemplary in nature and should not be used to limit the claims. Other embodiments are contemplated in accordance with the techniques described herein; such embodiments are within the scope of this application. Further, there is no intent to be bound by any expressed or implied theory presented in this application. The words “exemplary” or “illustrative” mean “serving as an example, instance, or illustration.” Any implementations described as “exemplary” or “illustrative” are not to be construed as preferred or advantageous over other implementations. All the implementations described below are exemplary implementations, in order to enable people skilled in the art to make or use the disclosed embodiments and are not intended to limit the scope of the disclosure, which is defined by the claims.

Some features can be described using relative terms, such as “upper,” “lower,” “left,” “rear,” “right,” “front,” “vertical,” “horizontal.” Such relative terms are only for reference with respect to the appended Figures and are not meant to limit the disclosed embodiments. Hence, specific dimensions and other physical characteristics relating to the disclosed embodiments are not meant to limit the claims, unless expressly stated otherwise in the claims.

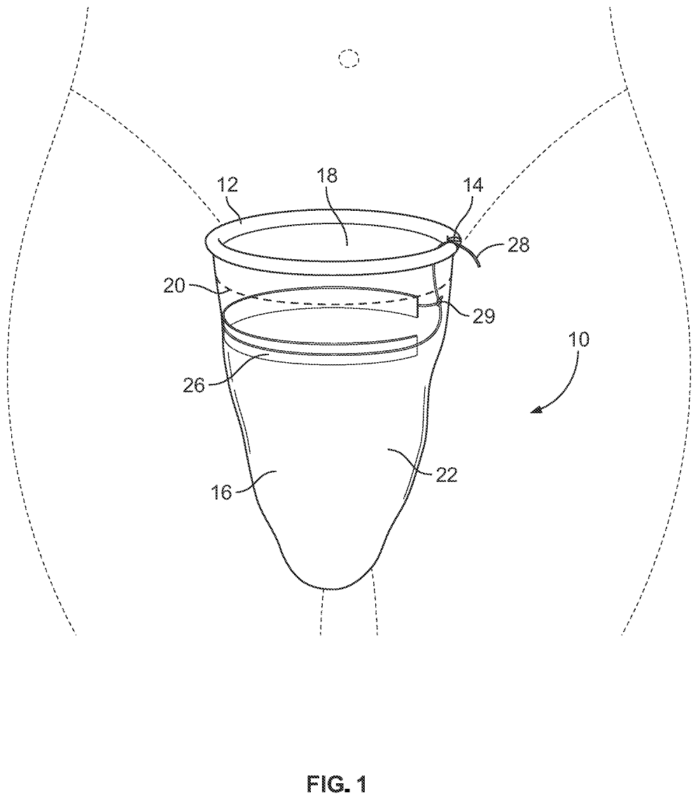

The diameter of the annular rim 12, as shown below in FIGS. 1-3B, can expand to the range of 25-85 mm after the annular rim 12, the film reservoir 16, and a portion of the drawstring 28 of the bodily fluid collection device 10 are placed within the vagina. The annular rim 12, shown below in FIGS. 1-3B can have a thickness ranging from 0-15 cm.

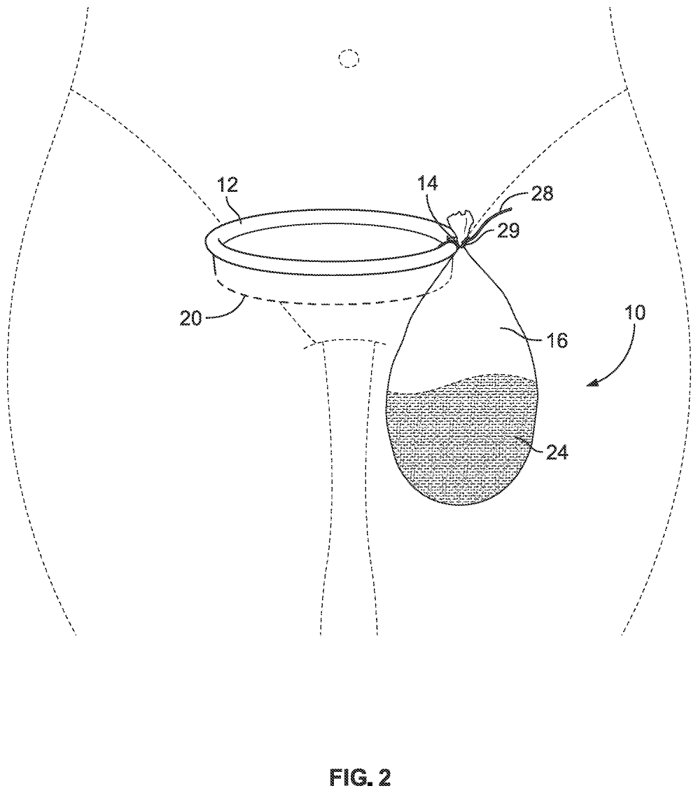

FIG. 1 illustrates a perspective view of the bodily fluid collection device 10. The bodily fluid collection device 10 can comprise an annular rim 12. The annular rim 12 can have a diameter that can range from 25-85 mm. An inner diameter of the annular rim 12 can have a diameter of 51 mm. An outer diameter of the annular rim 12 can have a diameter of 54 mm. The annular rim 12 can be made of a material comprising silicone, rubber, thermoplastic elastomers, variations thereof, or combinations thereof. In some aspects of the present disclosure, an opening 14 can be located on the annular rim 12. The opening 14 located on the annular rim 12 can be located in a vertical position, a horizontal position, or diagonally. A diameter of the opening 14 can be 0.6 mm. In some aspects of the disclosure, the diameter of the opening 14 can receive a drawstring 28. The drawstring 28 can be configured to seal the film reservoir 16. The bodily fluid collection device 10 can comprise a film reservoir 16 located below the annular rim 12. In other words, an annular rim 12 can be attached above a film reservoir 16. The film reservoir 16 can collect 0-150 ml of bodily fluids 24, as shown below in FIG. 2. In other words, a film reservoir 16 can receive a volume of bodily fluids. Bodily fluids 24 can comprise blood, menses, vaginal discharge, mucus, other biological discharge, or combinations thereof. The film reservoir 16 can expand or stretch as the film reservoir 16 collects the bodily fluids 24, as shown below in FIG. 2. In some aspects of the present disclosure, the film reservoir 16 can be flexible and malleable and not hold its shape unless it comprises a bodily fluid 24. In some aspects of the present disclosure, the film reservoir 16 can be semi-ridged and hold its shape. In some aspects of the present disclosure, the film reservoir 16 can have one or more perforations 20 circling a diameter of the film reservoir 16. The one or more perforations 20 can be located below the annular rim 12 and can be scored onto the film reservoir 16. The film reservoir 16 can be made of a material comprising nitrile, biodegradable nitriles, synthetic latex, lambskin, variants thereof, or combinations thereof.

In some aspects of the disclosure, a sleeve 26 can be located in an internal chamber 18 of the film reservoir 16, wherein the sleeve 26 can encircle a portion of the diameter of the internal chamber 18 of the film reservoir 16. In other aspects of the disclosure, the sleeve 26 can be located on an external face 22 of the film reservoir 16, wherein the sleeve 26 can encircle a portion of the diameter of the external face 22 of the film reservoir 16. The sleeve 26 can be located below the one or more perforations 20. In some embodiments, a space between the sleeve 26 and the annular rim 12 can comprise one or more perforations 20 circling the diameter of the film reservoir 16. A space between the sleeve 26 and the annular rim 12 can be an upper portion of the film reservoir 16. A space can also be considered an area. In some aspects of the disclosure, the sleeve 26 can be joined to the drawstring 28. In some aspects of the disclosure, the drawstring 28 can include a knot 29, wherein the knot 29 can be a slipknot. In some aspects of the disclosure, the knot 29 can be located in the internal chamber 18 of the film reservoir 16. In other aspects of the disclosure, the knot 29 can be located on the external face 22 of the film reservoir 16. The knot 29 can encompasses a portion of the diameter of the internal chamber 18 or the portion of the diameter of the external face 22 of the film reservoir 16 that the sleeve 26 does not encircle. A portion of the drawstring 28 can protrude from the human body, not shown. The portion of the drawstring 28 that can protrude from the human body can be the distal end of the drawstring 28 (not shown). The distal end of the drawstring 28 can be accessed when the bodily fluid collection device 10 is positioned for example, in a vagina or at the base of a cervix.

The drawstring 28 can be pulled to sever the film reservoir 16 from the annular rim 12, as shown below in FIG. 2, and prevent the bodily fluids 24 from escaping the film reservoir 16. In some aspects of the present disclosure, the drawstring 28 can sever the film reservoir 16 from the annular rim 12 at the one or more perforations 20. Thus, in some embodiments, the film reservoir 16 is partially removably attached to the annular rim 12. Partially removably attached can mean that a portion of the film reservoir 16 can remain attached to the annular rim 12, while another portion of the film reservoir 16 can be detached from the annular rim 12. In some aspects of the present disclosure, the sleeve 26 and the drawstring 28 can be pulled to constrict the film reservoir 16. Thus, in some embodiments, the constricted, sealed, cinched, or closed film reservoir 16 can remain entirely attached to the annular rim 12. In some aspects of the present disclosure, the knot 29 can be located at the opening 14 on the annular rim 12 and remain in the opening 14 on the annular rim 12 while the sleeve 26 and the drawstring 28 constrict the film reservoir 16. The knot 29 can seal the opening 14 on the annular rim 12 and can prevent the bodily fluids 24, shown below in FIG. 2, from escaping the film reservoir 16. The distal end of the drawstring 28 can be pulled and the drawstring 28 can guide the annular rim 12 and the film reservoir 16 out of the human body. In some aspects of the disclosure, the sleeve 26 can be made of a material comprising nitrile, biodegradable nitriles, synthetic latex, lambskin, variants thereof, or combinations thereof. The drawstring 28 can be made of nylon, polypropylene, variants thereof, or combinations thereof, and can have a length ranging from 0-75 cm.

FIG. 2 illustrates an alternate view of the bodily fluid collection device 10. The bodily fluid collection device 10 can comprise an annular rim 12. The annular rim 12 can have the diameter that can range between 25-85 mm. An inner diameter of the annular rim 12 can have a diameter of 51 mm. The outer diameter of the annular rim can have a diameter of 54 mm. The annular rim 12 can be made of a material comprising silicone, rubber, thermoplastic elastomers, variations thereof, or combinations thereof. In some aspects of the present disclosure, an opening 14 can be located on the annular rim 12. The opening 14 located on the annular rim 12 can be located in a vertical position, a horizontal position, or diagonally. A diameter of the opening 14 can be 0.6 mm. In some aspects of the disclosure, the diameter of the opening 14 can receive a drawstring 28. The bodily fluid collection device 10 can comprise a film reservoir 16 located below the annular rim 12. The film reservoir 16 can collect 0-150 ml of bodily fluids 24. The film reservoir 16 can expand or stretch to fit the human body as the film reservoir 16 collects the bodily fluids 24, as shown below in FIG. 2. In some aspects of the present disclosure, the film reservoir 16 can have one or more perforations 20 circling a diameter of the film reservoir 16. The one or more perforations 20 can be located below the annular rim 12 and can be scored onto the film reservoir 16. The film reservoir can be made of a material comprising nitrile, synthetic latex, lambskin, variants thereof, or combinations thereof. In some aspects of the disclosure, the drawstring 28 can have a knot 29, wherein the knot 29 can be a slipknot. A portion of the drawstring 28 can protrude from the human body, not shown. The portion of the drawstring 28 that can protrude from the human body can be the distal end of the drawstring 28 (not shown). The distal end of the drawstring 28 can be accessed when the bodily fluid collection device 10 is positioned for example, in a vagina or at the base of a cervix.

The drawstring 28 can be pulled to sever the film reservoir 16 from the annular rim 12 and prevent the bodily fluids 24 from escaping the film reservoir 16. In some aspects of the present disclosure, the drawstring 28 can sever the film reservoir 16 from the annular rim 12 at the one or more perforations 20. In some aspects of the present disclosure, the knot 29 can seal the opening 14 on the annular rim 12 and can assist the sleeve 26 and the drawstring 28 in constricting the film reservoir 16. By sealing the opening 14 on the annular rim 12, the knot 29 can prevent the bodily fluids 24 from escaping the film reservoir 16. The distal end of the drawstring 28 can be pulled and the drawstring 28 can guide the annular rim 12 and the film reservoir 16 out of the human body. The drawstring 28 can be made of nylon, polypropylene, variants thereof, or combinations thereof, and can have a length ranging from 0-75 cm.

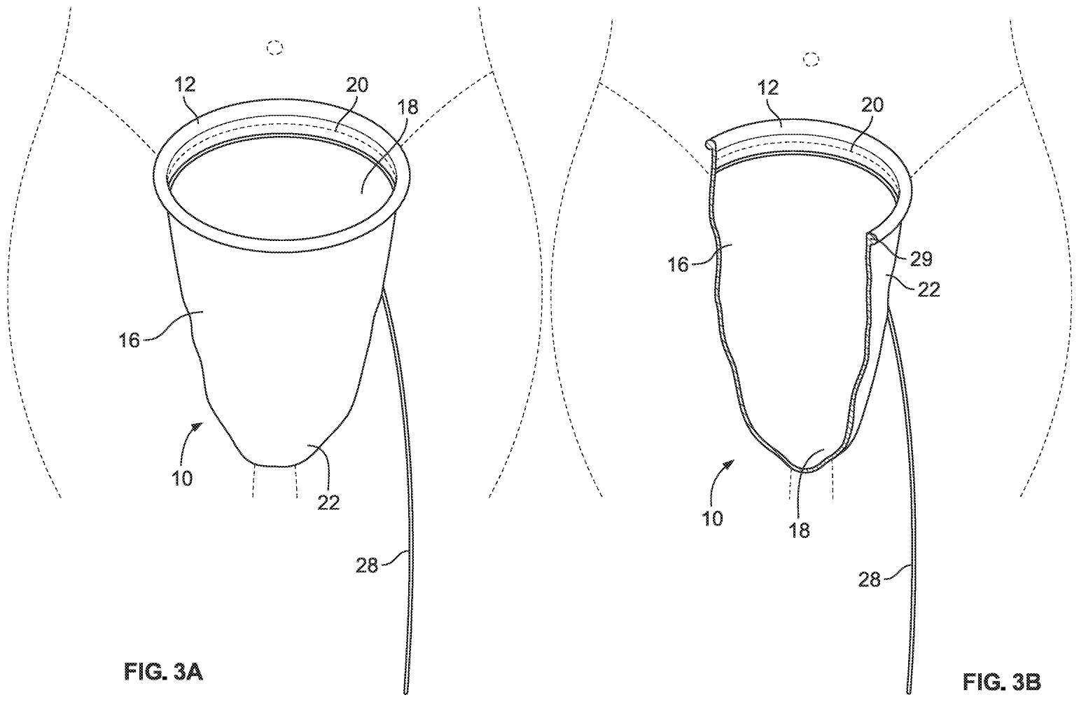

FIG. 3A illustrates an isometric view of the bodily fluid collection device 10. The bodily fluid collection device 10 can comprise the annular rim 12. The annular rim 12 can have the diameter that can range from 25-85 mm. The inner diameter of the annular rim 12 can be 51 mm. The outer diameter of the annular rim 12 can be 54 mm. The annular rim 12 can be made of a material comprising silicone, rubber, thermoplastic elastomers, variations thereof, or combinations thereof. The bodily fluid collection device 10 can comprise the film reservoir 16 located below the annular rim 12. In some aspects of the present disclosure, the film reservoir 16 can have one or more perforations 20 circling the diameter of the internal chamber 18 of the film reservoir 16. The one or more perforations 20 can be located below the annular rim 12 and can be scored onto the film reservoir 16. In some aspects of the disclosure, the drawstring 28 can be located below the one or more perforations 20 and encircle the diameter of the internal chamber 18 of the film reservoir 16. A portion of the drawstring 28 can protrude from the human body, not shown. The portion of the drawstring 28 that can protrude from the human body can be the distal end of the drawstring 28 (not shown). The distal end of the drawstring 28 can be accessed when the bodily fluid collection device 10 is positioned for example, in a vagina or at the base of a cervix. The drawstring 28 can be made of nylon, polypropylene, variants thereof, or combinations thereof, and can have a length ranging from 0-75 cm.

In some aspects of the disclosure, the bodily fluid collection device 10 can comprise a drawstring 28 that can be located below the annular rim. In some embodiments, the drawstring 28 can encircle the diameter of the external face 22 of the film reservoir 16. In some aspects of the disclosure, the drawstring 28 can comprise a knot 29, wherein the knot 29 can be a slipknot. In some aspects of the disclosure, the knot 29 can be located in the internal chamber 18 of the film reservoir 16. In some embodiments, the sleeve 26 (not shown in FIG. 3A-B) can be located below the one or more perforations 20. In some aspects of the disclosure, the sleeve 26 can be joined to the drawstring 28. In some embodiments, a sleeve 26 can house a drawstring 28. In other aspects of the disclosure, the knot 29 can be located on the external face 22 of the film reservoir 16. The knot 29 can encompasses a portion of the diameter of the internal chamber 18 or the portion of the diameter of the external face 22 of the film reservoir 16 that the sleeve 26 does not encircle.

FIG. 3B illustrates a cross-sectional isometric view of the bodily fluid collection device 10. The bodily fluid collection device 10 can comprise the annular rim 12. The annular rim 12 can have the diameter that can range from 25-85 mm. The inner diameter of the annular rim 12 can have a diameter of 51 mm. The outer diameter of the annular rim 12 can have a diameter of 54 mm. The annular rim 12 can be made of a material comprising silicone, rubber, thermoplastic elastomers, variations thereof, or combinations thereof. The bodily fluid collection device 10 can comprise a film reservoir 16 located below the annular rim 12. In some aspects of the present disclosure, the film reservoir 16 can have one or more perforations 20 circling the diameter of the film reservoir 16. The one or more perforations 20 can be located below the annular rim 12 and can be scored onto the film reservoir 16. In some aspects of the disclosure, the drawstring 28 can be located below the one or more perforations 20 and encircle the diameter of the internal chamber 18 of the film reservoir 16. In some aspects of the disclosure, the drawstring 28 can have a knot 29, wherein the knot 29 can be a slipknot. A portion of the drawstring 28 can protrude from the human body, not shown. The portion of the drawstring 28 that can protrude from the human body can be the distal end of the drawstring 28 (not shown). The distal end of the drawstring 28 can be accessed when the bodily fluid collection device 10 is positioned for example, in a vagina or at the base of a cervix. The drawstring 28 can be made of nylon, polypropylene, variants thereof, or combinations thereof, and can have a length ranging from 0-75 cm.

In some aspects of the disclosure, the bodily fluid collection device 10 can comprise a drawstring 28 that can be located below the annular rim. In some embodiments, the drawstring 28 can encircle the diameter of the external face 22 of the film reservoir 16. In some aspects of the disclosure, the drawstring 28 can comprise a knot 29, wherein the knot 29 can be a slipknot. In some aspects of the disclosure, the knot 29 can be located in the internal chamber 18 of the film reservoir 16. In some embodiments, the sleeve 26 (not shown in FIG. 3A-B) can be located below the one or more perforations 20. In some aspects of the disclosure, the sleeve 26 can be joined to the drawstring 28. In other aspects of the disclosure, the knot 29 can be located on the external face 22 of the film reservoir 16. The knot 29 can encompasses a portion of the diameter of the internal chamber 18 or the portion of the diameter of the external face 22 of the film reservoir 16 that the sleeve 26 does not encircle.

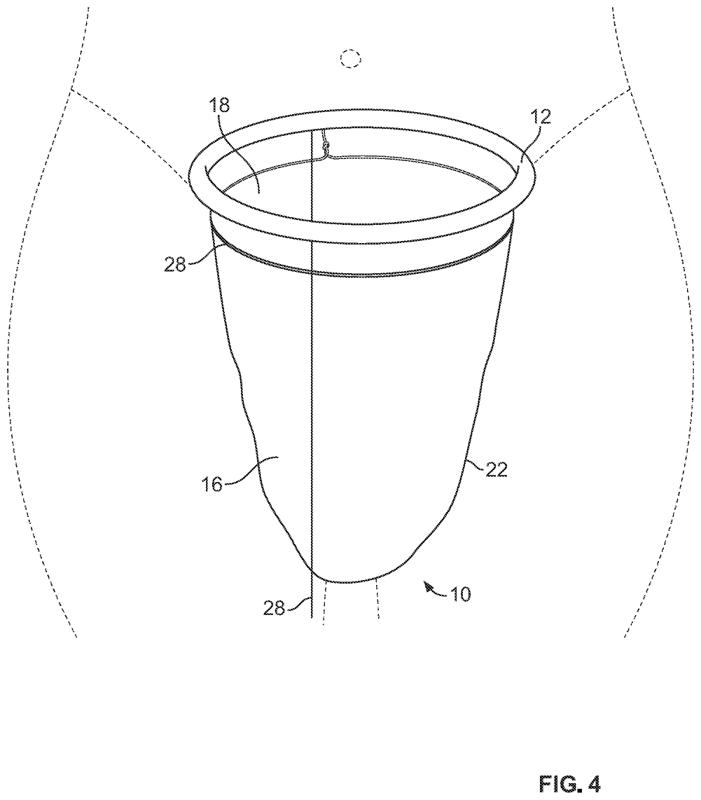

FIG. 4 is an isometric view of an exemplary embodiment of the bodily fluid collection device 10 not comprising perforations 20 as described in the present disclosure. The bodily fluid collection device 10 can comprise the annular rim 12. The annular rim 12 can have the diameter that can range from 25-85 mm. The inner diameter of the annular rim 12 can be 51 mm. The outer diameter of the annular rim 12 can be 54 mm. The annular rim 12 can be made of a material comprising silicone, rubber, thermoplastic elastomers, variations thereof, or combinations thereof. The bodily fluid collection device 10 can comprise the film reservoir 16 located below the annular rim 12. In some aspects of the present disclosure, the film reservoir 16 can be un-scored. Thus, in some embodiments, the film reservoir 16 can have no perforations 20 circling the diameter of the internal chamber 18 of the film reservoir 16, as shown in FIG. 4. In some aspects of the disclosure, the bodily fluid collection device 10 can comprise a drawstring 28 that can be located below the annular rim. In some embodiments, the drawstring 28 can encircle the diameter of the external face 22 of the film reservoir 16. In some aspects of the disclosure, the drawstring 28 can comprise a knot 29, wherein the knot 29 can be a slipknot. In some aspects of the disclosure, the knot 29 can be located in the internal chamber 18 of the film reservoir 16. In other aspects of the disclosure, the knot 29 can be located on the external face 22 of the film reservoir 16. The knot 29 can encompasses a portion of the diameter of the internal chamber 18 or the portion of the diameter of the external face 22 of the film reservoir 16. A portion of the drawstring 28 can protrude from the human body, not shown. The portion of the drawstring 28 that can protrude from the human body can be the distal end of the drawstring 28 (not shown). The distal end of the drawstring 28 can be accessed when the bodily fluid collection device 10 is positioned for example, in a vagina or at the base of a cervix. The drawstring 28 can be made of nylon, polypropylene, variants thereof, or combinations thereof, and can have a length ranging from 0-75 cm.

Method

Provided herein are methods for collecting bodily fluids. In some embodiments, the method for collecting bodily fluids can comprise inserting a bodily fluid collection device 10 into a vagina, wherein the bodily fluid collection device 10 can comprise a film reservoir 16 for receiving a bodily fluid 24, an annular rim 12 attached above the film reservoir 16, and a drawstring 28 configured to seal the film reservoir 16 collecting a volume of bodily fluids 24. The method can further comprise collecting a volume of bodily fluids 24 within the film reservoir 16; pulling a distal end of the drawstring 28 to seal the film reservoir 16 comprising a volume of bodily fluids 24 within the film reservoir 16; and removing the bodily fluid collection device 10 from an orifice with the film reservoir 16 in a closed state.

In some embodiments, the method can further comprise using an applicator to insert the bodily fluid collection device.

In some embodiments, the annular rim 12, film reservoir 16, drawstring 28, can be inserted into a body part, such as a vagina or base of cervix, and removed from an orifice, such as a vaginal opening, without needing to use fingers to insert or remove the device from the body part. In some embodiments, the annular rim 12, film reservoir 16, and drawstring 28 of the bodily fluid collection device 10 can be inserted into a body part such as a vagina or vaginal canal, and removed from an orifice, such as a vaginal opening manually, by hand, by depositing the bodily fluid collection device into the vagina, vaginal canal, or base of cervix and removing the bodily fluid collection device 10 with the film reservoir 16 in a sealed state using the distal end of the drawstring 28.

In some embodiments, pulling the distal end of the drawstring 28 to seal the film reservoir 16 comprising a volume of bodily fluids 24 within the film reservoir 16 comprises cinching the film reservoir 16 closed with the drawstring 28.

In some embodiments, pulling the distal end of the drawstring 28 to seal the film reservoir 16 comprising a volume of bodily fluids 24 within the film reservoir 16 comprises severing a portion of the film reservoir 16 with the drawstring 28, wherein pulling the drawstring 28 constricts the film reservoir 16.

In some embodiments, the drawstring 28 can be housed in a sleeve 26 affixed to an upper portion of the film reservoir 16. A sleeve 26 can be affixed, attached, integrally manufactured, or the like with an upper portion of the film reservoir 16. An upper portion of the film reservoir can be an area of the film reservoir 16 that is close to an annular rim 12.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter.

Although the disclosed subject matter has been described and illustrated in the foregoing illustrative embodiments, it is understood that the present disclosure has been made only by way of example, and that numerous changes in the details of implementation of the disclosed subject matter may be made without departing from the spirit and scope of the disclosed subject matter.

What is claimed is:

I claim:

1. A bodily fluid collection device, comprising:
a film reservoir for receiving a volume of bodily fluids;
an annular rim attached above the film reservoir; and
a drawstring configured to seal the film reservoir.

2. The bodily fluid collection device of claim 1, wherein the film reservoir is partially removably attached to the annular rim.

3. The bodily fluid collection device of claim 1, further comprising a sleeve affixed to an upper portion of the film reservoir, wherein the sleeve houses the drawstring.

4. The bodily fluid collection device of claim 3, wherein a space between the sleeve and the annular rim comprises perforations circling a diameter of the film reservoir.

5. The bodily fluid collection device of claim 1, wherein the annular rim has an opening that receives a portion of the drawstring.

6. The bodily fluid collection device of claim 1, wherein the drawstring includes a knot.

7. The bodily fluid collection device of claim 1, wherein the film reservoir expands and stretches while collecting bodily fluids.

8. The bodily fluid collection device of claim 1, wherein the bodily fluid collection device collects vaginal discharge.

9. A method of collecting bodily fluids, the method comprising:
inserting a bodily fluid collection device into a vagina, wherein the bodily fluid collection device comprises a film reservoir for receiving a bodily fluid, an annular rim attached above the film reservoir, and a drawstring configured to seal the film reservoir;
collecting a volume of bodily fluids within the film reservoir;
pulling a distal end of the drawstring to seal the film reservoir comprising the volume of bodily fluids within the film reservoir; and
removing the bodily fluid collection device from an orifice with the film reservoir in a closed state.

10. The method of claim 9, wherein pulling the distal end of the drawstring to seal the film reservoir comprising the volume of bodily fluids within the film reservoir comprises cinching the film reservoir closed with the drawstring.

11. The method of claim 9, wherein pulling the distal end of the drawstring to seal the film reservoir comprising a volume of bodily fluids within the film reservoir comprises severing a portion of the film reservoir with the drawstring, wherein pulling the drawstring constricts the film reservoir.

12. The method of claim 9, further comprising housing the drawstring in a sleeve affixed to an upper portion of the film reservoir.
